(19) United States
(12) Patent Application Publication
(10) Pub. No.: US 2014/0113284 A1
(43) Pub. Date: Apr. 24, 2014

(54) DIFFERENTIAL DETECTION AND QUANTIFICATION OF OXALOBACTER

(71) Applicants: Cuong Q. Nguyen, Gainesville, FL (US); Ammon B. Peck, Gainesville, FL (US)

(72) Inventors: Cuong Q. Nguyen, Gainesville, FL (US); Ammon B. Peck, Gainesville, FL (US)

(73) Assignee: UNIVERSITY OF FLORIDA RESEARCH FOUNDATION, INC., Gainesville, FL (US)

(21) Appl. No.: 13/882,853

(22) PCT Filed: Nov. 1, 2011
(86) PCT No.: PCT/US11/58808

§ 371 Date: Dec. 20, 2013

Related U.S. Application Data
(60) Provisional application No. 61/408,706, filed on Nov. 1, 2010.

(51) Int. Cl. C12Q 1/68

(52) U.S. Cl. CPC C12Q 1/689
USPC 435/6.11; 536/24.32

(57) ABSTRACT

Disclosed herein are PNA probes, or PNA probe sets and their use as well as kits useful for the analysis of certain Oxalobacter species and/or strain(s) present in a sample of interest. Probe sequences may sequence that are at least about 86% identical to the nucleobase sequence or complement thereof selected from the following sequences: GACAATGTAGAGTTGACT (SED ID NO. 1); caggatggtcagaagttc (SEQ ID NO. 2); CCGGTTACATCGAAGGA (SEQ ID NO. 3); and AATGTAGAGTTG ACT (SEQ ID NO. 4).

### CROSS-REFERENCE TO RELATED APPLICATIONS

This application is related to U.S. Provisional Application No. 61/408,706; filed Nov. 1, 2010, to which priority is claimed under 35 USC 119, and which is incorporated herein in its entirety.

### FIELD OF THE INVENTION

The present invention relates to peptide nucleic acid (PNA) probes, PNA probe sets and methods for the analysis of certain Oxalobacter species optionally present in a sample. The invention further relates to diagnostic kits comprising such PNA probes or PNA probe sets. The methods and kits of the invention are particularly useful for simultaneous analysis of one or more of Oxalobacter species or strain(s).

### BACKGROUND

Oxalobacter formigenes is a relatively recently discovered, oxalate-degrading obligately anaerobic bacterium residing primarily in the intestines of vertebrate animals, including man (Allison et al., 1986). Although the first isolates of O. formigenes were cultured from sheep rumen (Dawson et al., 1980), additional strains have now been isolated from fecal contents of rats, guinea pigs and pigs (Argenzio et al., 1988, Daniel et al., 1987), fecal samples from man (Allison et al., 1985), and anaerobic aquatic sediments (Smith et al., 1985). This bacterium is unique among oxalate-degrading organisms having evolved a total dependence on oxalate metabolism for energy (Dawson et al., 1980). Recent evidence suggests that Oxalobacter formigenes has an important symbiotic relationship with vertebrate hosts by regulating oxalic acid absorption in the intestine as well as oxalic acid levels in the plasma (Hatch and Freel, 1996). Studies by Jensen and Allison (1994) comparing various O. formigenes isolates revealed only limited diversity of their cellular fatty acids, proteins, and nucleic acid fragments. Based on these comparisons, strains of O. formigenes have been divided into two major subgroups. In general, group I strains have shown limited intragroup diversity, while group II strains have shown greater intragroup diversity.

While strides have been made in detecting the presence of Oxalobacter formigenes in humans, the inventors have surmised that improved identification and quantification of Oxalobacter species and/or strain(s) would be advantageous in many cases. Furthermore, the simultaneous analysis of multiple Oxalobacter strain(s) will be valuable in evaluating and identifying particularly beneficial strain(s) of Oxalobacter that can in turn be used therapeutically.

### SUMMARY

This invention is directed to PNA probes, or PNA probe sets and their use as well as kits useful for the analysis of certain Oxalobacter species and/or strain(s) present in a sample of interest.

In certain embodiments, the PNA probes are directed to ribosomal RNA (rRNA) or the genomic sequences corresponding to said rRNA (rDNA) or its complement.

In one embodiment, at least a portion of the probe is at least about 86% identical to the nucleobase sequence or complement thereof selected from the following sequences: GACAATGTAGAGTTGACT (SED ID NO. 1); CAGGATGGTCAGAAGTTC (SEQ ID NO. 2); CCGGTTACATCGAAGGA (SEQ ID NO. 3); and AATGTAGAGTTG ACT (SEQ ID NO. 4). SEQ ID NO. 4 represents a sequence that universally hybridizes to nucleic acid sequences from Oxalobacter species.

In another embodiment, at least a portion of the probe is selected from the following sequences: GACAATGTAGAGTTGACT (SED ID NO. 1); CAGGATGGTCAGAAGTTC (SEQ ID NO. 2); CCGGTTACATCGAAGGA (SEQ ID NO. 3); and AATGTAGAGTTG ACT (SEQ ID NO. 4).

In one embodiment, the probe sequence is between about 8-17 subunits in length. In one embodiment, the probe is labeled with at least one detectable moiety.

In one embodiment, the detectable moiety or moieties are selected from the group consisting of: a conjugate, a branched detection system, a chromophore, a fluorophore, a spin label, a radioisotope, an enzyme, a hapten, an acridinium ester and a luminescent compound.

In one embodiment, the probe is self-reporting. In another embodiment, the probe is a PNA Linear Beacon. In one embodiment, the probe is unlabeled. In one embodiment, the probe is bound to a support. In another embodiment, the probe further comprises a spacer or a linker.

In one embodiment, a probe includes the following components and sequence: FAM-OO-AAT GTA GAG TTG ACT, where FAM is the fluorescent molecule (6-carboxyfluorescein, or 6-FAM). The OO represent oxygen molecules. It has been discovered that positioning one or more oxygen molecules between the probe sequence and the fluorescent molecule (such as use as a linker) produces a more robust signal. It is believed that the oxygen linker ameliorates any internal interference with the fluorescent molecule.

In one embodiment, the probes are differently labeled for independent analysis of two or more Oxalobacter species and/or strain(s).

In one aspect, provided herein are PNA probe sets comprising one or more PNA probes and at least one PNA blocking probe for the analysis of one or more Oxalobacter species and/or strain(s).

In another embodiment, the probe sets further comprise a PNA blocking probe for analysis of Oxalobacter species and/or strain(s).

In one aspect, provided herein are methods for the analysis of Oxalobacter species and/or strain(s) in a sample, comprising: a) contacting at least one probe set to the sample, said probe set being specific to a target sequence of a Oxalobacter species and/or strain(s), b) hybridizing the PNA probe to a target sequence of Oxalobacter species and/or strain(s) in the sample; and c) detecting the hybridization, wherein the detection of hybridization is indicative of the presence, identity and/or amount of Oxalobacter species and/or strain(s) in the sample.

In another embodiment, the probes are independently detectable non-independently detectable, or a combination of independently and non-independently detectable, wherein the probes differ from one another by as little as a single base, and are complementary or substantially complementary to partially conserved target regions of phylogenetically related organisms.

In one embodiment, probes or probe sets are used to eliminate or reduce cross hybridization between a Oxalobacter species and/or strain(s) specific probe, and a separate Oxalobacter species and/or strain(s) target. In one embodiment, the analysis takes place in situ. In another embodiment, the analysis takes place by fluorescence in situ hybridization.

In one embodiment, the methods are used to detect a nucleic acid comprising a target sequence wherein said nucleic acid has been synthesized or amplified in a reaction. In one embodiment, preferred nucleic acid synthesis or nucleic acid amplification reactions are selected from the group consisting of: Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase.

In one embodiment, the methods further comprise adding at least one blocking probe to reduce or eliminate hybridization of the PNA probe to non-target sequence. In another embodiment, the target sequence is immobilized to a surface. In one embodiment, the PNA probe is immobilized to a surface. In one embodiment, the PNA probe is one component of an array. In one embodiment, the sample is a biological sample. In another embodiment, the biological sample is blood, urine, secretion, sweat, sputum, stool, mucous, or cultures thereof. In a more typical example, the biological sample comprises a stool sample.

In one aspect, provided herein are kits suitable for performing an assay for analysis of one or more Oxalobacter species and/or strain(s) in a sample, wherein said kit comprises: a) a PNA probe specific to an Oxalobacter species and/or strain(s) and b) other reagents or compositions necessary to perform the assay.

In one embodiment, two or more Oxalobacter species and/or strain(s) present in a sample are independently detected, identified and/or quantitated. In one embodiment, one or more Oxalobacter species and/or strain(s) present in a sample are independently detected, identified and/or quantitated.

In another embodiment, the kit is used in an in situ hybridization assay. In one embodiment, the kit is a fluorescence in situ hybridization assay for simultaneous, independent (multiplex) identification of one more Oxalobacter species and/or strain(s). In one embodiment, the kit is used for a real-time PCR assay.

In one embodiment, the kit is used to examine clinical samples such as clinical specimens or cultures thereof.

### BRIEF DESCRIPTION OF DRAWINGS

FIG. 1 relates to an image showing detection of Oxalobacter (green fluorescence) in a sample.

FIG. 2 relates to an image showing detection of Oxalobacter (green fluorescence) in a sample.

### DETAILED DESCRIPTION

### 1. Definitions

As used herein, the term “nucleobase” means those naturally occurring and those non-naturally occurring heterocyclic moieties commonly known to those who utilize nucleic acid technology or utilize peptide nucleic acid technology to thereby generate polymers that can specifically bind to nucleic acids.

As used herein, the term “nucleobase sequence” means any segment of a polymer that comprises nucleobase-containing subunits. Non-limiting examples of suitable polymers or polymer segments include oligodeoxynucleotides, oligoribonucleotides, peptide nucleic acids, nucleic acid analogs, nucleic acid mimics, and/or chimeras.

As used herein, the term “target sequence” means the nucleobase sequence that is to be detected in an assay.

As used herein, the term “probe” means a polymer (e.g. a DNA, RNA, PNA, chimera or linked polymer) having a probing nucleobase sequence that is designed to sequence-specifically hybridize to a target sequence of a target molecule of an organism of interest.

As used herein, the term “peptide nucleic acid” or “PNA” means any oligomer, linked polymer or chimeric oligomer, comprising two or more PNA subunits (residues), including any of the polymers referred to or claimed as peptide nucleic acids in U.S. Pat. Nos. 5,539,082, 5,527,675, 5,623,049, 5,714,331, 5,736,336, 5,773,571, 5,786,461, 5,837,459, 5,891,625, 5,972,610, 5,986,053, 6,107,470 and 6,357,163. In the most preferred embodiment, a PNA subunit consists of a naturally occurring or non-naturally occurring nucleobase attached to the aza nitrogen of the N-[2-(aminoethyl)]glycine backbone through a methylene carbonyl linkage.

As used herein, the terms “label” and “detectable moiety” are interchangeable and shall refer to moieties that can be attached to a probe to thereby render the probe detectable by an instrument or method.

Reference herein to “Oxalobacter species” refers to any bacteria classified under the genus of Oxalobacter. Reference to “Oxalobacter strain(s),” refers to an organism classified under a species of Oxalobacter, such as O. formigenes, but which possess gene sequences that are identifiably different from another organism classified under the same species. Appendix A shows examples of different strains of O. formigenes having sequence homologies and sequence differences. The strains in Appendix A are also categorized into two groups, group 1 and group 2, that share homologies as indicated (see shading Ox-1 and Ox-2). Also, both groups share homologies noted by shading (see all-Ox).

The term “sample” as used herein refers to any biological sample or clinical sample that could contain an analyte for detection. Examples of the biological sample, include but are not limited to, stool, urine, blood, wounds, sputum, laryngeal swabs, gastric lavage, bronchial washings, aspirates, serum, nasal discharge, sweat, plasma, semen, cerebrospinal fluid, tears, pus, amniotic fluid, saliva, lung aspirate, gastrointestinal contents, vaginal discharge, urethral discharge, expectorates and cultures thereof. Preferred tissue samples or cultures thereof include chorionic villi specimens, skin epithelials, genitalia epithelials, gum epithelials, throat epithelials, hair and biopsies. Tissue samples may be either freshly prepared, or preserved for some time in a fixative such as but not limited to ethanol, methanol, paraformaldehyde, glutaraldehyde, formalin, paraffin, formaldehyde, formamide, or mixtures thereof. Non-clinical samples include food, beverages, water, pharmaceutical products, personal care products, dairy products or environmental samples and cultures thereof.

As used herein, “multiplex assay” includes assays in which multiple targets can potentially be detected, and identified. Identification can be specific, e.g., a particular species of microorganism, or generic, e.g., a particular genus of microorganism. Generic identification may also include the identification of a cohort of species of microorganisms, which includes one or more members of a defined group of species.

As used herein, independent and simultaneous detection includes an assay that can at once yield identification results for more than one target. For example, the detection of one or more species or strains of Oxalobacter may be done at the same time with unique labels for each probe, thus resulting in independent and simultaneous detection.

Other reagents or compositions necessary to perform the assay, may include, for example, wash solutions, slides, coverslips, one or more PNA probes according to the invention, culture vessels, slide warmer, incubator, mounting fluid, and PCR components, including enzymes and buffers.

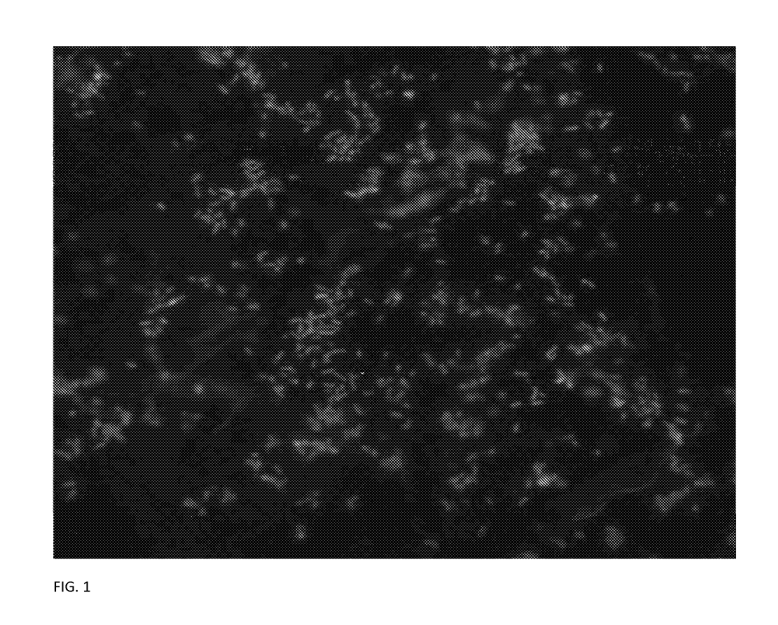

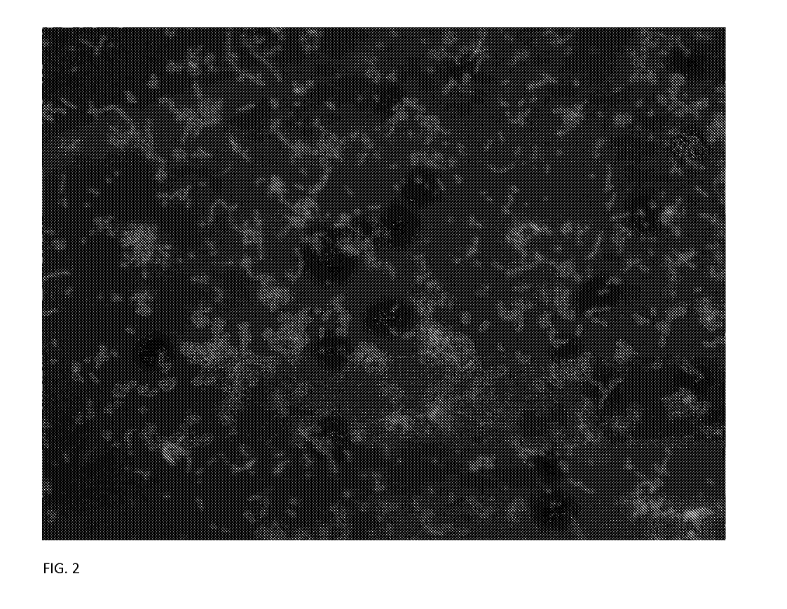

PNA probes (originally described in U.S. Pat. No. 5,539,082 and Egholm et al., Nature 365:566-568 (1993), herein attached as reference) have inherent physico/chemical characteristics as compared to naturally occurring nucleic acid probes, which allow the design of rapid and accurate assays. PNA probes offer another advantage over nucleic acid probes when applied in fluorescence in situ hybridization (FISH) assays due to their improved cellular penetration of the rigid cell wall of Gram-positive bacteria. Where nucleic acid probes require fixation and permeabilization with cross-linking agents and/or enzymes (for example see Kempf et al., J. Clin. Microbiol 38:830-838 (2000)), PNA probes can be applied directly following smear preparation. FIGS. 1 and 2 represent images produced by a FISH assay performed on two different samples O. formigenes that were set on a slide. FIGS. 1 and 2 utilized the FAM-OO-AAT GTA GAG TTG ACT probe.

In preferred embodiments, PNA probes have relatively short nucleobase sequences. Naturally occurring nucleic acid probes due to their weaker stabilities and lower melting temperatures (Tm) are typically at least 18 nucleobases in length (For example see Kempf et al., J. Clin. Microbiol 38:830-838 (2000)). The greater specificity of PNA probes provides better discrimination to closely related non-target sequences with a single or just a few nucleobase difference(s) as required for analysis of rRNA or rDNA of closely related Oxalobacter species and/or strain(s).

Exemplary PNA probe nucleobase sequences according to the invention include: GACAATGTAGAGTTGACT (SED ID NO. 1); CAGGATGGTCAGAAGTTC (SEQ ID NO. 2); CCGGTTACATCGAAGGA (SEQ ID NO. 3) or AATGTAGAGTTG ACT (SEQ ID NO. 4).

In yet another embodiment, the PNA probes may be part of a PNA probe set comprising either two or more PNA probes for analysis of two or more species or strains of Oxalobacter species. That is, some PNA probes of the invention are specific for two or more species of Oxalobacter species and/or strain(s). Preferably, PNA probes within a PNA probe set are differently labeled for independent analysis of two or more Oxalobacter species and/or strain(s). In a modification of this embodiment, multiple probes may be identically labeled to detect a particular Oxalobacter species or cohort of Oxalobacter species, while an optional other probe or probes is differently labeled for analysis of a second species or cohort of species.

The method according to the invention comprises contacting a sample with one or more of the PNA probes described above. According to the method, the presence, absence and/or number of Oxalobacter species and/or strain(s) are detected, identified and/or quantitated by correlating the hybridization, under suitable hybridization conditions, of the probing nucleobase sequence of the probe to the target sequence. Consequently, the analysis is based on a single assay with a definitive outcome.

Different probes may be used either in parallel reactions or in the same reaction (multiplex) for simultaneous analysis of Oxalobacter species and/or strain(s). This way the presence of Oxalobacter species and/or strain(s) are further supported by the absence of an Oxalobacter specific signal or visa versa, such final test results are interpreted on the basis on both a positive and a negative reaction. The PNA probe set therefore provides internal controls which eliminate the need to perform separate control experiments. Preferably, the two PNA probes are independently labeled such that the analysis is performed in one reaction (multiplex). Simultaneous analysis is also an advantage for specimens containing a mixture of Oxalobacter species and/or strain(s). In such cases the use of the PNA probes for FISH offers the advantage of single cell detection, such that cells of Oxalobacter species and/or strain(s), and O. formigenes can be viewed simultaneously and differentiated by specific labels as exemplified below. In contrast other technologies are not able to distinguish mixed cultures from false-positive reactions without performing additional control experiments.

In still another embodiment, this invention is directed to kits suitable for performing an assay that detects, identifies and/or quantitates Oxalobacter species and/or strain(s) optionally present in a sample and/or determination of antibiotic resistance. The kits of this invention comprise one or more PNA probes and other reagents or compositions that are selected to perform an assay or otherwise simplify the performance of an assay. In particular, the combined analysis of Oxalobacter species and/or strain(s) is well-suited for routine testing of stool samples where the use of multiple PNA probes serve secondarily as internal controls.

Another benefit derived from the use of multiple PNA probes is the use of blocking probes. In this preferred embodiment, the blocking probe strategy is employed in the design of probes for use in a multiplex assay where probes are directed against similar regions of conserved target molecules in closely related organisms. For example, a pair of independently detectable probes which are substantially similar in nucleobase sequence and Tm may be designed to hybridize to highly conserved regions of two targets in which the targeted nucleobase sequences differ by only one base. In this case, the tendency of the first probe to hybridize non-specifically to the complementary target of the second probe is discouraged by the presence and relative stability of the second probe/target hybrid. Similarly, multiple probes may be designed which are either independently detectable or non-independently detectable, or a combination of independently and non-independently detectable, which all differ from one another by as little as a single base, and which are complementary, or at least substantially complementary to partially conserved target regions of phylogenetically related organisms. The competition for target sites can result in higher probe specificities, while lowering the likelihood of cross hybridization.

Those of ordinary skill in the art will appreciate that a suitable PNA probe need not have exactly these probing nucleobase sequences described herein to be operative but may be modified according to the particular assay conditions. For example, shorter PNA probes can be prepared by truncation of the nucleobase sequence if the stability of the hybrid needs to be modified to thereby lower the Tm and/or adjust for stringency. Similarly, the nucleobase sequence may be truncated at one end and extended at the other end as long as the discriminating nucleobases remain within the sequence of the PNA probe. Such variations of the probing nucleobase sequences within the parameters described herein are considered to be embodiments of this invention.

Those of ordinary skill in the art will also appreciate that the complement probing sequence is equally suitable for assays, such as but not limited to real-time PCR, that are directed against rDNA as a target sequence.

PNA Synthesis:

Methods for the chemical assembly of PNAs are well known (see: U.S. Pat. Nos. 5,539,082, 5,527,675, 5,623,049, 5,714,331, 5,736,336, 5,773,571, 5,786,461, 5,837,459, 5,891,625, 5,972,610, 5,986,053 and 6,107,470).

PNA Labeling:

Preferred non-limiting methods for labeling PNAs are described in U.S. Pat. Nos. 6,110,676, 6,361,942, and 6,355,421, the examples section of these specifications or are otherwise well known in the art of PNA synthesis and peptide synthesis.

Labels:

Non-limiting examples of detectable moieties (labels) suitable for labeling PNA probes used in the practice of this invention would include a dextran conjugate, a branched nucleic acid detection system, a chromophore, a fluorophore, a spin label, a radioisotope, an enzyme, a hapten, an acridinium ester and a chemiluminescent compound.

Other suitable labeling reagents and preferred methods of attachment would be recognized by those of ordinary skill in the art of PNA, peptide or nucleic acid synthesis.

Preferred haptens include 5 (6)-carboxyfluorescein, 2,4-dinitrophenyl, digoxigenin, and biotin.

Preferred fluorochromes (fluorophores) include 5 (6)-carboxyfluorescein (Flu), 6-carboxyfluorescein (6-FAM), tetramethyl-6-carboxyrhodamine (tamra), 6-((7-amino-4-methylcoumarin-3-acetyl)amino) hexanoic acid (Cou), 5 (and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.), JOE, Tamara or the Alexa dye series (Molecular Probes, Eugene, Oreg.).

Preferred enzymes include polymerases (e.g. Taq polymerase, Klenow PNA polymerase, T7 DNA polymerase, Sequenase, DNA polymerase 1 and phi29 polymerase), alkaline phosphatase (AP), horseradish peroxidase (HRP) and most preferably, soy bean peroxidase (SBP).

Unlabeled Probes:

The probes that are used for the practice of this invention need not be labeled with a detectable moiety to be operable within the methods of this invention, for example, the probes of the invention may be attached to a solid support, which renders them detectable by known array technologies.

Self-Indicating Probes:

Beacon probes are examples of self-indicating probes which include a donor moiety and an acceptor moiety. The donor and acceptor moieties operate such that the acceptor moieties accept energy transferred from the donor moieties or otherwise quench signal from the donor moiety. Though the previously listed fluorophores (with suitable spectral properties) might also operate as energy transfer acceptors, preferably, the acceptor moiety is a quencher moiety. Preferably, the quencher moiety is a non-fluorescent aromatic or heteroaromatic moiety. The preferred quencher moiety is 4-((-4-(dimethylamino)phenyl)azo)benzoic acid (dabcyl). In a preferred embodiment, the self-indicating Beacon probe is a PNA Linear Beacon as more fully described in U.S. Pat. No. 6,485,901.

In another embodiment, the self-indicating probes of this invention are of the type described in WIPO patent application WO97/45539. These self-indicating probes differ as compared with Beacon probes primarily in that the reporter must interact with the nucleic acid to produce signal.

Spacer/Linker Moieties:

Generally, spacers are used to minimize the adverse effects that bulky labeling reagents might have on hybridization properties of probes. Preferred spacer/linker moieties for the nucleobase polymers of this invention consist of one or more aminoalkyl carboxylic acids (e.g., aminocaproic acid), the side chain of an amino acid (e.g., the side chain of lysine or omithine), natural amino acids (e.g., glycine), aminooxyalkylacids (e.g., 8-amino-3,6-dioxaoctanoic acid), alkyl diacids (e.g., succinic acid), alkyloxy diacids (e.g., diglycolic acid) or alkyldiamines (e.g., 1,8-diamino-3,6-dioxaoctane).

Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

Suitable Hybridization Conditions:

Generally, the more closely related the background causing nucleic acid contaminants are to the target sequence, the more carefully stringency must be controlled. Blocking probes may also be used as a means to improve discrimination beyond the limits possible by optimization of stringency factors. Suitable hybridization conditions will thus comprise conditions under which the desired degree of discrimination is achieved such that an assay generates an accurate (within the tolerance desired for the assay) and reproducible result.

Aided by no more than routine experimentation and the disclosure provided herein, those of skill in the art will easily be able to determine suitable hybridization conditions for performing assays utilizing the methods and compositions described herein. Suitable in situ hybridization or PCR conditions comprise conditions suitable for performing an in situ hybridization or PCR procedure. Thus, suitable in situ hybridization or PCR conditions will become apparent to those of skill in the art using the disclosure provided herein, with or without additional routine experimentation.

Blocking Probes:

Blocking probes are nucleic acid or non-nucleic acid probes that can be used to suppress the binding of the probing nucleobase sequence of the probing polymer to a non-target sequence. Preferred blocking probes are PNA probes (see: U.S. Pat. No. 6,110,676). It is believed that blocking probes operate by hybridization to the non-target sequence to thereby form a more thermodynamically stable complex than is formed by hybridization between the probing nucleobase sequence and the non-target sequence. Through formation of the more stable and preferred complex, the less stable non-preferred complex between the probing nucleobase sequence and the non-target sequence is prevented from forming. Thus, blocking probes can be used with the methods, kits and compositions of this invention to suppress the binding of the probes to a non-target sequence that might be present and interfere with the performance of the assay.

Probing Nucleobase Sequence:

The probing nucleobase sequence of a probe of this invention is the specific sequence recognition portion of the construct. Therefore, the probing nucleobase sequence is a nucleobase sequence designed to hybridize to a specific target sequence wherein the presence, absence or amount of the target sequence can be used to directly or indirectly detect the presence, absence or number of organisms of interest in a sample. Consequently, with due consideration to the requirements of a probe for the assay format chosen, the length and sequence composition of the probing nucleobase sequence of the probe will generally be chosen such that a stable complex is formed with the target sequence under suitable hybridization conditions.

The preferred nucleobase sequences of the probes of this invention for analysis of Oxalobacter species and/or strain(s) are SEQ ID NOs 1-3, and/or the complements thereof.

This invention contemplates that variation in these identified probing nucleobase sequences shall also provide probes that are suitable for the analysis of Oxalobacter species and/or strain(s). Such variation of the probing nucleobase sequences within the parameters described herein are considered to be an embodiment of this invention. Common variations include, deletions, insertions and frame shifts. Additionally, a shorter probing nucleobase sequence can be generated by truncation of the sequences identified above.

A probe of this invention will generally have a probing nucleobase sequence that is exactly complementary to the target sequence. Alternatively, a substantially complementary probing nucleobase sequence might be used since it has been demonstrated that greater sequence discrimination can be obtained when utilizing probes wherein there exists one or more point mutations (base mismatch) between the probe and the target sequence (See: Guo et al., Nature Biotechnology 15:331-335 (1997)). Consequently, the probing nucleobase sequence may be only as much as 86% homologous to the probing nucleobase sequences identified above. Substantially complementary probing nucleobase sequences within the parameters described above are considered to be an embodiment of this invention.

Complements of the probing nucleobase sequence are considered to be an embodiment of this invention, since it is possible to generate a suitable probe if the target sequence to be detected has been amplified or copied to thereby generate the complement to the identified target sequence.

Detection, Identification and/or Enumeration:

By detection is meant analysis for the presence or absence of the organism optionally present in the sample. By identification is meant establishment of the identity of the organism by genus name, by genus and species name, or by other suitable category which serves to classify the organism(s) of interest. By quantitation is meant enumeration of the organisms in a sample. Some assay formats provide simultaneous detection, identification and enumeration (for example see Stender, H. et al., J. Microbiol. Methods. 45:31-39 (2001), others provide detection and identification (for example see Stender, H. et al., Int. J. Tuberc. Lung Dis. 3:830-837 (1999) and yet other assay formats just provide identification (for example see Oliveira, K et al. J. Clin. Microbiol. 40:247-251 (2002)).

Independent and Simultaneous Detection:

In a preferred embodiment of this invention, a multiplex assay is designed to detect one target with a labeled probe, while simultaneously detecting another target with a differently labeled probe. In a more preferred embodiment of the invention, the targets constitute rRNA or rDNA from two or more Oxalobacter species and/or strain(s). In the most preferred embodiment, the assay is a PNA FISH assay designed to detect one or species of Oxalobacter species and/or strain(s).

Antibiotic Resistance

By determination of resistance to antibiotics is meant analysis of an organism's susceptibility to antibiotics based on specific genes or gene products, or mutations associated with resistance or susceptibility to antimicrobial agents.

### II. Preferred Embodiments of the Invention

a. PNA Probes:

In one embodiment, this invention is directed to PNA probes. The PNA probes of this invention are suitable for detecting, identifying and/or quantitating Oxalobacter species and/or strain(s) optionally present in a sample. General characteristics (e.g., length, labels, nucleobase sequences, linkers, etc.) of PNA probes suitable for the analysis have been previously described herein. The preferred probing nucleobase sequence of PNA probes of this invention pertain to SEQ ID NOs. 1-3 or fragments thereof, or fragments of the sequences provided in Appendix A.

The PNA probes of this invention may comprise only a probing nucleobase sequence (as previously described herein) or may comprise additional moieties. Non-limiting examples of additional moieties include detectable moieties (labels), linkers, spacers, natural or non-natural amino acids, or other subunits of PNA, DNA or RNA. Additional moieties may be functional or non-functional in an assay. Generally however, additional moieties will be selected to be functional within the design of the assay in which the PNA probe is to be used. The preferred PNA probes of this invention are labeled with one or more detectable moieties selected from the group consisting of fluorophores, enzymes and haptens.

In preferred embodiments, the probes of this invention are used in in situ hybridization (ISH) and fluorescence in situ hybridization assays. Excess probe used in an ISH or FISH assay typically must be removed so that the detectable moiety of the specifically bound probe can be detected above the background signal that results from still present but unhybridized probe. Generally, the excess probe is washed away after the sample has been incubated with probe for a period of time. However, the use of self-indicating probes is a preferred embodiment of this invention, since there is no requirement that excess self-indicating probe be completely removed (washed away) from the sample since it generates little or no detectable background. In addition to ISH or FISH assays, self-indicating probes comprising the selected probing nucleobase sequence described herein are particularly useful in all kinds of homogeneous assays such as in real-time PCR or useful with self-indicating devices (e.g. lateral flow assay) or self-indicating arrays.

b. PNA Probe Sets

Probe sets of this invention comprise two or more PNAs. In one embodiment, some of the PNA probes of the set can be blocking probes. In other embodiments, the probe set can be used to analyze two of more Oxalobacter species and/or strain(s), or for the analysis of one or more Oxalobacter species and/or strain(s).

c. Methods:

In another embodiment, this invention is directed to a method suitable for analysis of Oxalobacter species and/or strain(s) optionally in a sample. The general and specific characteristics of PNA probes suitable for the analysis of Oxalobacter species and/or strain(s) have been previously described herein.

The method for analysis of Oxalobacter species and/or strain(s) in a sample comprises contacting the sample with one or more PNA probes suitable for hybridization to a target sequence which is specific to Oxalobacter species and/or strain(s). According to the method, Oxalobacter species and/or strain(s) in the sample is then detected, identified and/or quantitated or its resistance to antibiotics is determined. This is made possible by correlating hybridization, under suitable hybridization conditions or suitable in situ hybridization conditions, of the probing nucleobase sequence of a PNA probe to the target sequence of Oxalobacter species and/or strain(s) sought to be detected with the presence, absence or number of the Oxalobacter species and/or strain(s) organisms in the sample. Typically, this correlation is made possible by direct or indirect detection of the probe/target sequence hybrid. In a preferred embodiment, a PNA probe set is used for simultaneous analysis of Oxalobacter species and/or strain(s) using differently labeled PNA probes.

Fluorescence in situ Hybridization and Real-time PCR:

The PNA probes, methods, kits and compositions of this invention are particularly useful for the rapid probe-based analysis of Oxalobacter species and/or strain(s), preferably using PNA probe sets for simultaneous analysis of two or more Oxalobacter species and/or strain(s). In preferred embodiments, in situ hybridization or PCR is used as the assay format for analysis of Oxalobacter species and/or strain(s). Most preferably, fluorescence in situ hybridization (PNA FISH) or real-time PCR is the assay format (Reviewed by Stender et al. J. Microbiol. Methods 48:1-17 (2002)). Preferably, smears for PNA FISH analysis are not treated with cross-linking agents or enzymes prior to hybridization.

In one embodiment, the method includes synthesizing a nucleic acid from a sample, for example by nucleic acid amplification. Preferred nucleic acid amplification reactions are selected from the group consisting of: Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.). Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/USO1/04285, which are all incorporated herein by reference in their entirety for all purposes. Patents that describe synthesis techniques in specific embodiments include U.S. Pat. Nos. 5,412,087, 6,147,205, 6,262,216, 6,310,189, 5,889,165, and 5,959,098. For PCR methods see, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1,17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference. Nucleic acid arrays that are useful in the present invention include those that are commercially available from Affymetrix (Santa Clara, Calif.). Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86,1173 (1989) and WO88/10315), self-sustained sequence replication (Guatelli et al., Proc. Nat. Acad. Sci. USA, 87,1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA, see, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11,1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80:1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

Exemplary Assay Formats:

Exemplary methods for performing PNA FISH can be found in: Oliveira et al., J. Clin. Microbiol 40:247-251 (2002), Rigby et al., J. Clin. Microbiol. 40:2182-2186 (2002), Stender et al., J. Clin. Microbiol. 37:2760-2765 (1999), Perry-O'Keefe et al., J. Microbiol. Methods 47:281-292 (2001). According to one method, a smear of the sample, such as, but not limited to, a positive blood culture, is prepared on microscope slides and covered with one drop of the fluorescent-labeled PNA probe in hybridization buffer. A coverslip is placed on the smear to ensure even coverage, and the slide is subsequently placed on a slide warmer or incubator at 55 degrees C. for 90 minutes. Following hybridization, the coverslip is removed by submerging the slide into a pre-warmed stringent wash solution and the slide is washed for 30 minutes. The smear is finally mounted with one drop of mounting fluid, covered with a coverslip and examined by fluorescence microscopy.

Oxalobacter species and/or strain(s) optimally present in a sample which may be analyzed with the PNA probes contained in the kits of this invention can be evaluated by several instruments, such as but not limited to the following examples: microscope (for example see Oliveira et al., J. Clin. Microbiol 40:247-251 (2002)), radiation sensitive film (for example see Perry-O'Keefe et al., J. Appl. Microbiol. 90:180-189) (2001), camera and instant film (for example see Stender et al., J. Microbiol. Methods 42:245-253 (2000)), luminometer (for example see Stender et al., J. Microbiol. Methods. 46:69-75 (2001), laser scanning device (for example see Stender et al., J. Microbiol. Methods. 45: 31-39 (2001) or flow cytometer (for example see Wordon et al., Appl. Environ. Microbiol. 66:284-289 (2000)). Automated slide scanners and flow cytometers are particularly useful for rapidly quantitating the number of microorganisms present in a sample of interest.

Exemplary methods for performing real-time PCR using self-reporting PNA probes can be found in: Fiandaca et al., Abstract, Nucleic Acid-Based technologies, DNA/RNA/PNA Diagnostics, Washington, D.C., May 14-16, 2001, and Perry-O'Keefe et al., Abstract, International Conference on Emerging Infectious Diseases, Atlanta, 2002.

d. Kits:

In yet another embodiment, this invention is directed to kits suitable for performing an assay, which analyzes Oxalobacter species and/or strain(s) optionally present in a sample. The general and preferred characteristics of PNA probes suitable for the analysis of Oxalobacter species and/or strain(s) have been previously described herein. Furthermore, methods suitable for using PNA probes to analyze Oxalobacter species and/or strain(s) in a sample have been previously described herein.

The kits of this invention comprise one or more PNA probes and other reagents or compositions which are selected to perform an assay or otherwise simplify the performance of an assay used to analyze Oxalobacter species and/or strain(s) in a sample. In preferred embodiments, the kit comprises a PNA probe set for simultaneous analysis of Oxalobacter species and/or strain(s) using independently detectable PNA probes.

e. Exemplary Applications for Using the Invention:

The PNA probes, methods and kits of this invention are particularly useful for the analysis of Oxalobacter species and/or strain(s) in clinical samples, e.g., stool, urine, blood, wounds, sputum, laryngeal swabs, gastric lavage, bronchial washings, biopsies, aspirates, expectorates as well as in food, beverages, water, pharmaceutical products, personal care products, dairy products or environmental samples and cultures thereof. In preferred embodiments, the PNA probes are applied in a PNA probe set also containing a PNA probe for analysis of Oxalobacter species and/or strain(s).

All references cited herein are incorporated in their entirety to the extent not inconsistent with the teaching herein. Having described the preferred embodiments of the invention, it will now become apparent to one of skill in the art that other embodiments incorporating the concepts described herein may be used. It is felt, therefore, that these embodiments should not be limited to disclosed embodiments but rather should be limited only by the spirit and scope of the following claims.

1. A PNA probe comprising a nucleobase sequence suitable for the analysis of one or more Oxalobacter species or strain(s).

2. A PNA probe comprising a nucleobase sequence suitable for the analysis of two or more Oxalobacter species or strain(s).

3. The PNA probe of claim 1, wherein a target sequence of the Oxalobacter species comprises rRNA, rDNA or a complement of rRNA or rDNA.

4. The PNA probe of claim 1, wherein the nucleobase sequence suitable for the analysis of Oxalobacter species, comprises one or more of a PNA probe complementary to a target sequence of the Oxalobacter species rRNA or rDNA or a complement thereof.

5. The PNA probe of claim 1, wherein the nucleobase sequence suitable for the analysis of Oxalobacter species, comprises a PNA probe complementary to a target sequence of Oxalobacter species rRNA or rDNA or a complement thereof.

6. The PNA probe of claim 1 wherein at least a portion of the probe is at least about 86% identical to the nucleobase sequence or complement thereof selected from SEQ ID NO. 1, 2 or 3.

7. The PNA probe of claim 1, wherein at least a portion of the probe is selected from the following sequences: GACAATGTAGAGTTGACT (SEQ ID NO. 1); CAGGATGGTCAGAAGTTC (SEQ ID NO. 2); CCGGTTACATCGAAGGA (SEQ ID NO. 3) or AATGTAGAGTTG ACT (SEQ ID NO. 4).

8. The PNA probe of claim 1, wherein the probe sequence is between about 8 and about 17 subunits in length.

9. The PNA probe of claim 1 wherein the probe is labeled with at least one detectable moiety.

10. A kit for detecting Oxalobacter bacteria in a biological sample, which comprises one or more PNA probes and a support, the PNA probes being immobilized on the support, wherein the PNA probes are capable of specifically binding with an Oxalobacter species and/or strain.

11. A method for detecting Oxalobacter species and/or strain(s) in a biological sample, which comprises the steps of: (a) adding a reaction sample containing a target DNA to the kit according to claim 10; (b) subjecting PNA probe(s) in the kit and the target DNA to hybridization; and (c) detecting the signal from the hybridization of PNA and; DNA.

12. A method for the analysis of one or more Oxalobacter species in a biological sample, said method comprising: a) contacting a PNA probe set to the sample, b) hybridizing the PNA probes to a target sequence of Oxalobacter species in the sample; and c) detecting the hybridization, wherein the detection of hybridization is indicative of the presence, identity and/or amount of Oxalobacter species in the sample.

13. The method of claim 12, wherein the probes are independently detectable non-independently detectable, or a combination of independently and non-independently detectable, wherein the probes differ from one another by as little as a single base, and are complementary or substantially complementary to partially conserved target regions of phylogenetically related organisms.

14. The method of claim 12, wherein an amount of Oxalobacter species is determined in said biological sample.

15. The method of claim 14, wherein said amount is determined via counting cells hybridized to said probes via flow cytometry.

16. The method of claim 14, wherein said amount is determined via a fluorescent signal.

17. The method of claim 12, wherein said probe set comprises a first probe hybridizable to a first target sequence, said first target sequence specific to a first Oxalobacter strain(s) and a second probe hybridizable to second target sequence, said second target sequence specific to a second Oxalobacter strain(s).

18. The method of claim 17, wherein said first probe comprises a first fluorescent tag and said second probe comprises a second fluorescent tag.

19. The method of claim 12, wherein the PNA probe is FAM-OO-AAT GTA GAG TTG ACT (SEQ ID NO: 4).

20. The method of claim 12, comprising performing a FISH assay.


SEQUENCE LISTING (4 sequences)

SEQ ID NO: 1 - DNA, 18 bp, Artificial Sequence
  Description of Artificial Sequence Synthetic oligonucleotide
gacaatgtagagttgact

SEQ ID NO: 2 - DNA, 18 bp, Artificial Sequence
  Description of Artificial Sequence Synthetic oligonucleotide
caggatggtcagaagttc

SEQ ID NO: 3 - DNA, 17 bp, Artificial Sequence
  Description of Artificial Sequence Synthetic oligonucleotide
ccggttacatcgaagga

SEQ ID NO: 4 - DNA, 15 bp, Artificial Sequence
  Description of Artificial Sequence Synthetic oligonucleotide
aatgtagagttgact